(19) United States
(12) Patent Application Publication
(10) Pub. No.: US 2019/0069920 A1
(43) Pub. Date: Mar. 7, 2019

(54) DEVICE FOR CLEAN EXCISION OF A HEART VALVE

(71) Applicant: UNIVERSITÉ CATHOLIQUE DE LOUVAIN, Louvain-la-Neuve (BE)

(72) Inventors: Parla Astarci, Kraainem (BE); Xavier Bollen, Woluwé-Saint-Lambert (BE); Khanh Tran Duy, Walhain (BE); Benoît Raucent, Wavre (BE)

(73) Assignee: UNIVERSITÉ CATHOLIQUE DE LOUVAIN, Louvain-la-Neuve (BE)

(21) Appl. No.: 16/084,269

(22) PCT Filed: Mar. 14, 2017
(86) PCT No.: PCT/EP2017/055901

§ 371 Date: Sep. 12, 2018

(30) Foreign Application Priority Data
Mar. 14, 2016 (EP) 16160156.2

(51) Int. Cl. A61B 17/3207; A61B 17/32

(52) U.S. Cl. CPC A61B 17/32075; A61B 2017/22097; A61B 17/320016

(57) ABSTRACT

A device (100) is presented for excision of a heart valve comprising a first (120) and second (140) clamping element in mutual sliding relation, each having an annular clamping surface (122, 142) which annular clamping surfaces (122, 142) mutually co-operate to form an annular clamping region (166) configured for clamping a heart valve annularly, and a slidable cutting element (160) slidable and rotatable with respect to the annular clamping region (166) configured to circularly excise the heart valve, wherein the slidable cutting element (160) is displaceable within an annulus of the annular clamping zone region (166).

### FIELD OF THE INVENTION

A device for the excision of a heart valve is presented.

### BACKGROUND TO THE INVENTION

The surgical procedure for excision of a heart valve involves cutting and removing the heart valve to form a circular aperture in which the replacement valve is inserted. Conventional devices require the application of high forces to cut through calcified tissue. Furthermore, the resulting aperture is not precisely located or dimensioned because the cutting edge is not stably positioned during cutting. In addition, the surgeon must ensure that debris generated during cutting is recovered.

An ordinary adaptation of a conventional venous cutter does not suffice. A venous cutter typically is used to create a valveless venous vessel for a venous bypass. Venous valves are not calcified; they are soft structures removed by shaving from the vessel inner surface. WO 2011/010296 discloses a device for a venous valvulotomy. It provides a cylindrical cutting tool that is drawn through the venous lumen, and basket-like elements disposed a fixed distance from the cutting tool for capture of the removed valves. U.S. Pat. No. 3,837,345 discloses a venous valve cutter having sharp spikes that spear and impale the venous valve leaflets; a secondary cutter clips the impaled spikes.

The presently described device aims to overcome the problems of the art.

### SUMMARY OF FEATURES

The present invention relates to a device (100) for excision of a heart valve comprising:
a first (120) and second (140) clamping element in mutual sliding relation, each having an annular clamping surface (122, 142) which annular clamping surfaces (122, 142) mutually co-operate to form an annular clamping region (166) configured for clamping a heart valve annularly, and
a slidable cutting element (160) slidable with respect to the annular clamping region (166) configured to circularly excise the heart valve,

wherein the slidable cutting element (160) is displaceable within an annulus of the annular clamping zone region (166).

The slidable cutting element (160) may further be rotatable with respect to the annular clamping region (166). The second clamping element (140) may comprise a cap (146), which cap (146) comprises a void space configured for retention of tissue debris. The first clamping element (120) may comprise a hollow tubular member (121). The slidable cutting element (160) and second clamping element (140) may mutually co-operate to form a first closed container for retention of tissue debris. The first (120) and second (140) clamping elements may mutually co-operate to form a second closed container for retention of tissue debris, wherein the first container is disposed within the second container. One of the first (120) or second (140) clamping elements may be configured to fittingly receive at least part of the other of the first (120) or second (140) clamping elements. The slidable cutting element (160) may be disposed on a circular edge of a cup-shaped body configured for retention of tissue debris. The first clamping element (120) may be attached to a first elongated tube (124), the slidable cutting element (160) is attached to a second elongated tube (164) arranged within a lumen of the first elongated tube (124), and the second clamping element (140) is attached to a longitudinal member (144) arranged within a lumen of the second elongated tube (164).

The device (100) may further comprise a heart valve balloon catheter (240) for deployment of an expandable heart valve (260).

The first clamping element (120) may be attached to a first elongated tube (124), the slidable cutting element (160) is attached to a second elongated tube (164) arranged within a lumen of the first elongated tube (124), and the second clamping element (140) is attached to a longitudinal member (144) arranged within a lumen of the second elongated tube (164), and the heart valve balloon catheter (240) may be substantially disposed within a lumen of the longitudinal member (144).

The first (120) and second (140) clamping elements and the slidable cutting element (160) may be each expandable in a radial dimension. In particular, they may be expandable in a radial dimension for retraction into a lumen of a deployment catheter (180). The first (120) and second (140) clamping elements and the slidable cutting element (160) may be each radially expandable compliant members biased in an open configuration. The device (100) may further comprise a deployment catheter (180), wherein the first (120) and second (140) clamping elements and the slidable cutting element (160) are retractable into a lumen of the deployment catheter (180).

The first (120) and second (140) clamping elements and the slidable cutting element (160) may be each radially non-expandable. The first (120) and second (140) clamping elements and the slidable cutting element (160) may be controllably radially expandable, optionally the respective radii being lockable.

### FIGURE LEGENDS

FIG. 1 is a longitudinal cross-sectional view of a device presented herein, where the first and second clamping elements have a fixed radial dimension and are in an open non-clamping configuration.

FIG. 2 is a longitudinal cross-sectional view of a device of FIG. 1, where the first and second clamping elements are in a closed clamping configuration.

FIG. 3A is a longitudinal cross-sectional view of a device presented herein for delivery through a catheter, where the first and second clamping elements have a reducible radial dimension, are in a deployable state and are in an open non-clamping configuration.

FIG. 3B is a longitudinal cross-sectional view of a device of FIG. 3A, wherein the first and second clamping elements and the slidable cutting element are in a withdrawn, non-deployable state.

FIG. 4 is a longitudinal cross-sectional view of a device of FIG. 3A, further provided with a balloon catheter for delivery of a heart valve.

FIG. 5 is a longitudinal cross-sectional view of a device of FIG. 4, further provided with a centering balloon catheter.

FIG. 6 is a plan view of an unfolded conical body used for instance in first or second clamping element or in a slidable cutting element.

FIG. 7 is a plan view of an unfolded conical body used for instance in first or second clamping element or in a slidable cutting element, show in detail are pivoting holes.

FIG. 8A is a photograph of a distal end of a device wherein the first and second clamping elements form a first closed container.

FIG. 8B is a photograph of a distal end of a device wherein the second clamping element and slidable cutting element have been advanced distally, and debris captured in the second closed container.

FIG. 8C is a photograph of a distal end of a device wherein the second clamping element and slidable cutting element have been separated, and substantially the whole heart valve is captured in the first closed container.

FIG. 9 is a longitudinal cross-sectional view of a device presented herein, where the first second clamping has a fixed radial dimension and second clamping element is radially foldable.

FIGS. 10A to 10D show a sequence of advancing the device of FIG. 9 through a heart valve, and locking the second clamping element in an open configuration.

FIG. 11 is a longitudinal cross-sectional view of a device disposed with a cylindrical cutting element.

### DETAILED DESCRIPTION OF INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. It is to be understood that other embodiments may be utilised and structural or logical changes may be made without departing from the scope of the present invention. The following detailed description, therefore, is not to be taken in a limiting sense, and the scope of the present invention is defined by the appended claims.

The terms “distal”, “distal end”, “proximal” and “proximal end” are used through the specification, and are terms generally understood in the field to mean towards (proximal) or away (distal) from the surgeon side of the apparatus. Thus, “proximal (end)” means towards the surgeon side and, therefore, away from the patient side. Conversely, “distal (end)” means towards the patient side and, therefore, away from the surgeon side. In the drawings, the proximal part of an element is indicated with reference sign (20) and the distal part of an element is indicated with reference sign (30).

The present invention concerns a device for excision of a heart valve. The device is a medical device or a surgical tool. The device is suitable for excision via a percutaneous route (e.g. transapical, transfemoral, transaortic routes) or by open heart surgery. The percutaneous route refers to accessing the heart valve via the vasculature, while the open heart surgery refers to surgically opening the heart. The heart valve may be any, for instance, a native diseased valve or a prosthetic valve. The device comprises a first (120) and second (140) clamping element in mutual sliding relation; the first clamping element (120) may be fixed while the second (140) clamping element is slidable in relation therewith, or vice versa, or both first (120) and second (140) clamping element may be slidable. Each of the first (120) and second (140) clamping elements has an annular clamping surface (122, 142) which annular clamping surfaces mutually co-operate to form an annular clamping region (166) configured for clamping a heart valve annularly. The first (120) and second (140) clamping elements, more particularly, the first (122) and second (142) annular clamping surfaces are disposed either side of the heart valve, are drawn together so as to annularly clamp the heart valve. The device further comprises a slidable cutting element (160) that is slidable with respect to the annular clamping region (166) and is configured for circularly excising the heart valve. The slidable cutting element (160) is displaceable within the annulus of the annular clamping zone. The annular clamping region (166) is disposed towards the distal end (30) of the device. The first clamping element (120) may be disposed proximal to the second clamping element (140). A radial dimension of the annular clamping region corresponds to the size of the excision.

The device of the invention provides an annular clamping region (166) for gripping heart valve. The inventors have realised that providing a mechanism for clamping the valve that is separate from a cutting mechanism increases the cutting efficiency. The annular clamping region (166) that surrounds the heart valve produces a valve tautness as the slidable cutting element advances. The tension created allows the valve to be more amenable to fast and clean release by the cutting edge (162) compared an absence of tension accordingly less cutting force is required. Concomitantly, the diameters of the force-transmitting components can be reduced so leading to a lighter and reduced-profile device. Additionally, there is a reduction in debris as the valve is more cleanly cut.

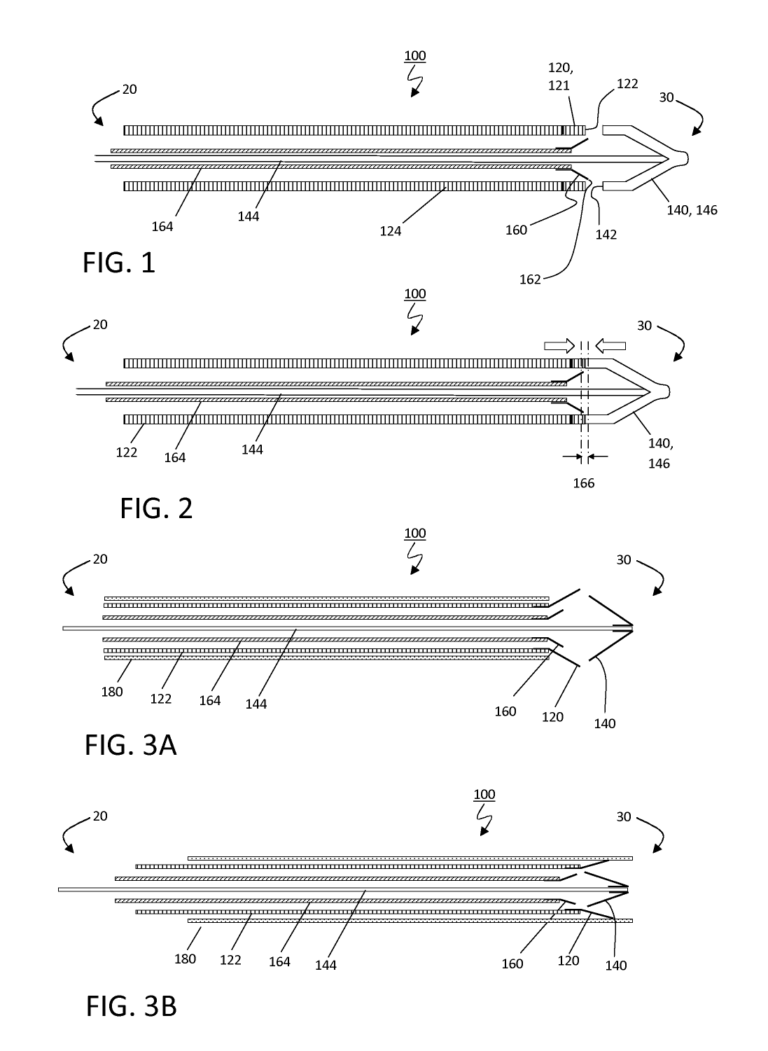

The second clamping element (140) may have the form of a cap (e.g. FIGS. 1 and 2) whereby a circular edge or rim of the cap forms the second annular clamping surface (142). The cap (146) comprises a void space configured for retention of tissue debris. The cap (146) preferably has an open end on which the second annular clamping surface (142) is disposed. The cap (146) open end preferably faces a proximal (20) direction. At the other end of the cap (146) is a closed end that preferably points in a distal (30) direction.

The second (140) clamping element may be elongate. The outer shape of second (140) clamping element preferably may be at least partly conical, most preferably frustoconical. The wide base of the cone preferably provides the open end, while the tip or truncated tip forms the closed end of the second (140) clamping element. The wide base of the cone also provides the second annular clamping surface (142). Other outer shapes are envisaged for instance, cylindrical, barrel, bullet, rivet and the like.

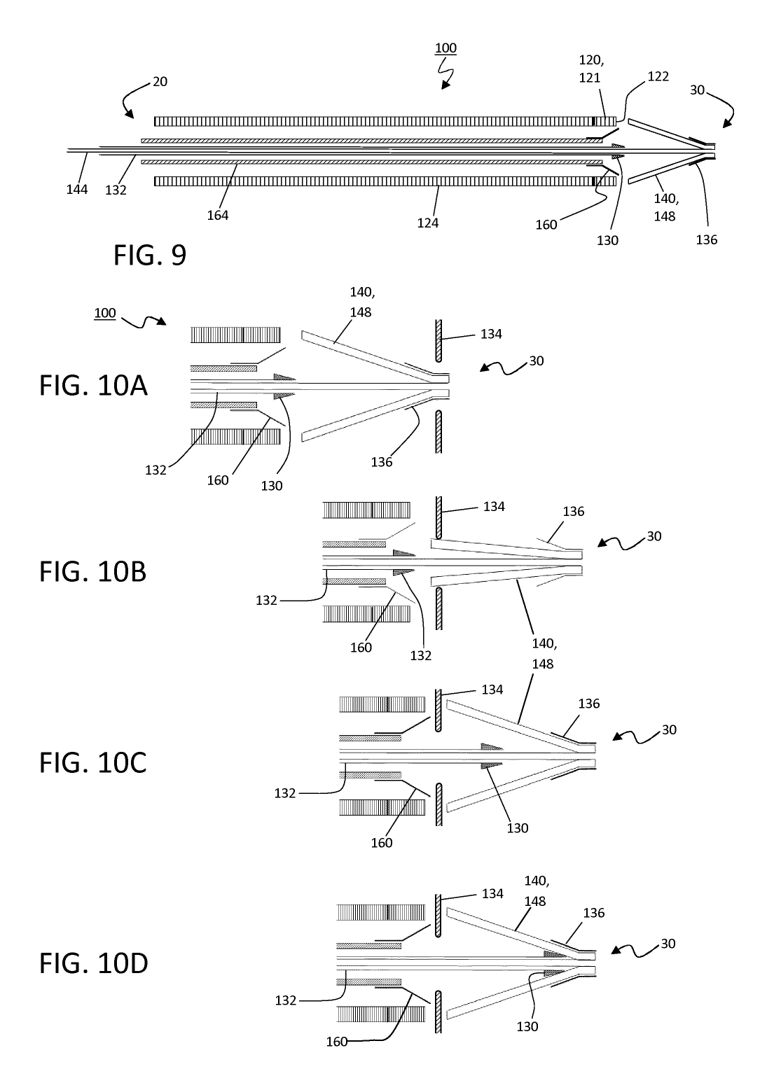

The second clamping element (140, 148) may be radially foldable from an open to a closed configuration. It is preferably radially compliant and biased in an open configuration. FIG. 9 depicts an exemplary device (100) provided with a radially compliant second clamping element (140, 148) biased in an open configuration. The radius of the second clamping element (140, 148) may be reduced upon the application of an external force acting in a radial direction. Being radially foldable the second clamping element (140, 148) may be advanced through a narrow passage of the heart valve (134) without substantial hindrance, and without creating additional debris. It is particularly suited for entry by open heart surgery. FIGS. 10A to 10C depict a sequence where a device (100) is advanced though a heart valve (134). In FIG. 10A, the distal tip (30) passes through without serious obstruction, and in FIG. 10B a restricting force applied to the second clamping element (140, 148) radially folds it. In FIG. 10C the second clamping element (140, 148) has passed through the heart valve (134); in the absence of the restricting force, it radially expands back to its native state. The outer shape of the radially expandable second clamping element (140, 148) may be at least partly conical, most preferably frustoconical.

The radial dimension of the second clamping element (140, 148) may be lockable in an open configuration. It may be locked in an open configuration by applying a locking force from the inside of the second clamping element (140, 148), for instance, from a slidable rigid body (130) having, for example a conical, frustoconical, cylindrical, barrel, bullet, or rivet and the like shape. FIGS. 10C to 10D depict a sequence wherein the second clamping element (140, 148) having advanced though a heart valve (134) is locked in the open configuration. The slidable rigid body (130) attached to a third elongated tube (132) is pushed distally, contacting and applying force to the second clamping element (140, 148) and locking it open. An expansion limiter (136) disposed in fixed slidable relation to the second clamping element (140, 148) limits the extent of expansion. The expansion limiter (136) preferably has a conical or frustoconical form that accommodates a conical shape of the second clamping element (140, 148).

The second clamping element (140) body may contain fine apertures or slots or it may be lined with a fine mesh for the passage of fluid and retention of tissue debris. As described later below the second annular clamping surface (142) or second clamping element (140) may have a fixed radial dimension (e.g. for entry by open heart surgery) or may be capable of radial expansion from a closed to an open configuration (e.g. for percutaneous route). The second annular clamping surface (142) or second clamping element (140) may be slidable relative to a handle disposed at the proximal end of the device (100). One or more radio-opaque markers provided on the second clamping element (140), preferably at a fixed distance from the second annular clamping surface (142).

The first clamping element (120) may have the form of a tubular member (121), preferably cylindrical, and the first clamping surface (122) may be disposed on a circular edge of the tubular member (121). The tubular member (121) may be a hollow tube disposed with a lumen defined by a wall. The tubular member (121) may or may not have a uniform radial dimension in a longitudinal direction (e.g. it may be uniform cylinder or frustoconical cylinder). The first clamping surface (122) may be defined by an edge of the wall of the tubular member (121), preferably at the distal end (30) of the tubular member (121). The tubular member (121) may extend towards the proximal end (20) of the device. The first clamping element (120) or tubular member (121) may have a fixed radial dimension.

Alternatively, the distal end of the first clamping element (120) or tubular member (121) may be capable of radial expansion from a closed to an open configuration (e.g. for percutaneous route). The first clamping element (120) body may contain fine apertures or slots or it may be lined with a fine mesh for the passage of fluid and retention of tissue debris. The first clamping element (120) or tubular member (121) may be slidably fixed relative to a handle disposed at the proximal end of the device (100). One or more radio-opaque markers may be provided on the first clamping element (120) or tubular member (121), preferably at a fixed distance from the first annular clamping surface (122).

The first clamping element (120) may be attached to or extend into a first elongated tube (124). A proximal end of the first clamping element (120) may be attached to a distal end of first elongated tube (124). The first elongated tube (124) may be a hollow tube disposed with a lumen defined by a wall. The lumen of the tubular member (121) may be in fluid connection with the lumen of the tubular member (124) or first clamping element (120). The first elongated tube (124) may extend towards the proximal end (20) of the device. The length of the first elongated tube (122) can depend on the route of entry, for instance, it will be longer for a device configured for percutaneous access (e.g. 1 to 3 m) via the vasculature compared with via open heart surgery (e.g. 15 to 40 cm).

The first (122) and second (142) annular clamping surfaces preferably have a similar shape e.g. circular. The may or may not have the same size. One of first (122) or second (142) annular clamping surfaces may be smaller than the other, thereby allowing one to be fitting received by the other; the second closed container (see below) so formed has greater integrity and stability, and the heart valve is made more taut during clamping.

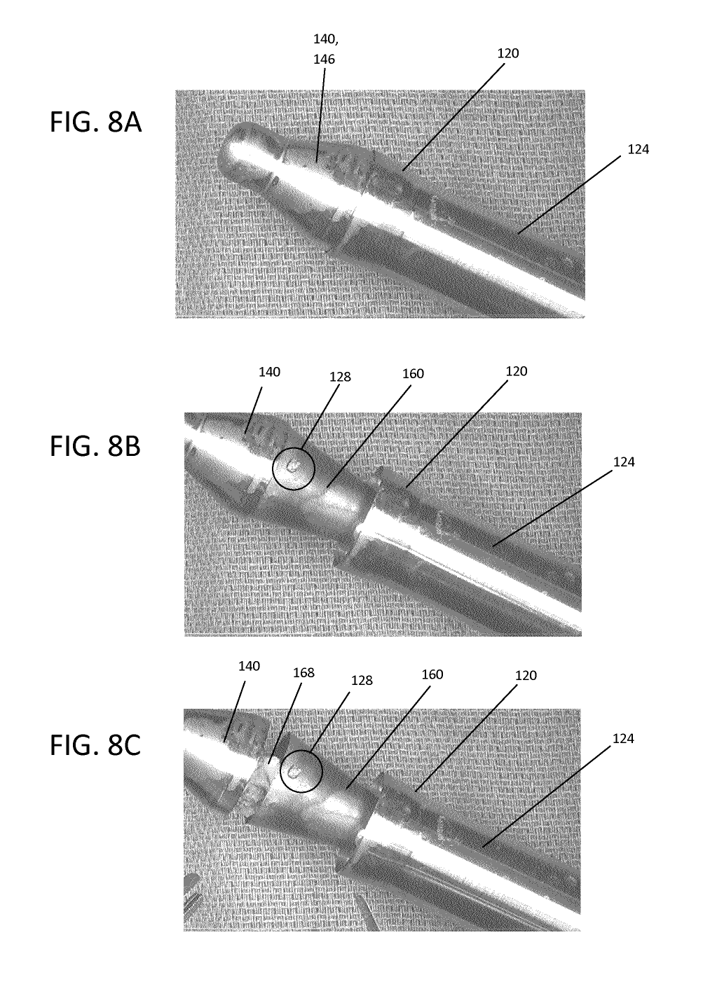

The slidable cutting element (160) and second clamping element (140) after cutting engage and couple to form a first closed container. The first closed container is formed after cutting by the slidable cutting element (160). It is appreciated that the respective walls of the slidable cutting element (160) and second clamping element (140) may each contain fine apertures or slots or they may each be lined with a fine mesh for the passage of fluid and retention of tissue debris. The first closed container is configured to contain the cut tissue debris. The inventors have found that, in practice, the first closed container contains the majority of tissue debris, namely most of the heart valve as shown, for instance, in FIG. 8C, where the excised heart valve (168) is indicated.

The first clamping element (120) and second clamping element (140) couple at the annular clamping region (166) to form a second closed container. The second closed container is formed prior to and during cutting by the slidable cutting element. It is appreciated that the respective walls of the slidable cutting element (160) and second clamping element (140) may each contain fine apertures or slots or they may each be lined with a fine mesh for the passage of fluid and retention of tissue debris. The second closed container is configured to contain cut tissue debris. The inventors have found that not all debris from cutting is contained within the first closed container. Surprisingly, tissue particles are found outside the cutting ring of the slidable cutting element. The second closed container captures additional debris not captured in the cap (146) FIG. 8A shows the device (100) after excision, and the formation of second closed container by first clamping element (120) and second clamping element (140). FIG. 8B shows the additional debris (128) captured by the second closed container.

The first closed container is disposed within the second closed container.

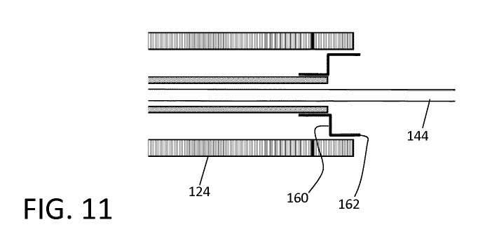

The slidable cutting element (160) comprises a cutting edge (162) for excision of the heart valve. The cutting edge (162) preferably has a circular profile. The slidable cutting element (160) comprises a body having void space configured for retention of tissue debris. The void space is dimensioned to capture the excised heart valve. The slidable cutting element (160) preferably comprises a conical or frustoconical form, as shown, for instance, in FIGS. 1 to 5. Other outer shapes are envisaged for instance, cylindrical (FIG. 11), barrel, bullet, rivet and the like. The slidable cutting element (160) body may contain fine apertures or slots or it may be lined with a fine mesh for the passage of fluid and retention of tissue debris.

The slidable cutting element (160) preferably has an open end for entry into the void space. The cutting edge (162) is disposed on the edge of the open end. The slidable cutting element (160) open end preferably faces a distal (30) direction. At the other end of the slidable cutting element (160) is a closed end that preferably points in a proximal (20) direction. One or more radio-opaque markers provided on the slidable cutting element (160), preferably at a fixed distance from the cutting edge (162).

The slidable cutting element (160) is displaceable within the annulus of the annular clamping region (166); the slidable cutting element (160) is displaceable within the closed container formed by the first clamping element (120) and second clamping element (140). The slidable cutting element (160) may be slidable relative to the first clamping element (120) and/or second clamping element (140). The slidable cutting element (160) may be rotatable about a central axis, more in particular. The slidable cutting element (160) may be rotatable within the annulus of the annular clamping region (166); the slidable cutting element (160) may be rotatable within the closed container formed by the first clamping element (120) and second clamping element (140). The slidable cutting element (160) may be rotatable relative to the first clamping element (120) and/or second clamping element (140). The annular clamping region (166) may be rotationally fixed relative to the first (120) and second (140) clamping elements. The first (120) and second (140) clamping elements may be mutually rotationally fixed (e.g. non rotatable), preferably in the annular clamping region (166).

To perform the cutting function, the cutting edge (162) may be sharpened. Additionally or alternatively, it may be disposed with an abrasive or cutting material such as diamond or graphite. Alternatively, or in addition, the cutting edge (162) may be jagged e.g. it may have teeth, triangular, square or otherwise. Preferably, the cutting edge (162) is designed to minimise the amount of debris produced. Preferably, the cutting edge (162) is designed to reduce the particle size of debris produced, so that it can be better retained or stored in the void space. The slidable cutting element (160) is configured for a cutting action which may be a rotation (continuous, intermittent, mono- or bi-directional, or alternative), a linear movement, a combination of these.

The slidable cutting element (160) may be configured for rotation around an axis that is preferably its central (longitudinal) axis. Rotation of the slidable cutting element (160) provides a rotating blade at the cutting edge (162) which results in a more efficient excision that may require less force compared with merely punching-out the defective heart valve.

It will be appreciated that other cutting actions, besides rotation, can be utilised, such as a linear displacement in a longitudinal direction. For instance, the cutting action may be an oscillation in the longitudinal direction that rapidly advances and withdraws the cutting edge, to provide a hammering action. It will be appreciated that the rotation and hammering action may be combined.

As described later below the slidable cutting element (160) or cup-shaped body (162) may have a fixed radial dimension (e.g. for entry by open heart surgery) or may be capable of radial expansion from a closed to an open configuration (e.g. for percutaneous route). The slidable cutting element (160) or cup-shaped body (162) may be slidable relative to a handle disposed at the proximal end of the device (100).

The first (120) and second (140) clamping elements are in mutual sliding relation. The second (140) clamping element may slide i.e. be displaceable relative to the first (120) clamping element. The second (140) clamping element may attached to a longitudinal member (144) that extends towards the proximal end of the device (100). The longitudinal member (144) is configured for the transmission of a displacement force from the proximal end (20) to the distal end (30) of the device (100). Thus the second (140) clamping element may be displaced relative to the first (120) responsive actuation of the longitudinal member (144) at the proximal end (20) of the device (100). The longitudinal member (144) at the proximal end (20) may be actuated manually by the surgeon or alternatively robotically. The longitudinal member (144) may be disposed within a lumen of the first elongated tube (122). The longitudinal member (144) may be disposed within a lumen of the second elongated tube (164). The longitudinal member (144) may be provided with a lumen for a guidewire or for a heart valve balloon catheter (200).

The slidable cutting element (160) is slidable and optionally rotatable relative to the annular clamping region (166). The slidable cutting element (160) may slide i.e. be displaceable relative to the annular clamping region (166). Preferably the slidable cutting element (160) is slidable and optionally rotatable relative to the first clamping element (120) or first elongated tube (122). The slidable cutting element (160) may be attached to a second elongated tube (162) that extends towards the proximal end of the device (100). The second elongated tube (162) is a hollow tube disposed with a longitudinal lumen defined by a wall. The second elongated tube (162) is configured for the transmission of a displacement force from the proximal end (20) to the distal end (30) of the device (100). Thus the second elongated tube (162) may be displaceable relative to the annular clamping region (166), more particularly relative to the first elongated tube (122), responsive actuation of the second elongated tube (162) at the proximal end (20) of the device (100). The second elongated tube (162) at the proximal end (20) may actuated manually by the surgeon or alternatively robotically.

The second elongated tube (162) may be further configured for the transmission of torque from the proximal end (20) to the distal end (30) of the device (100) such that rotation of the cutting edge (162) can be actuated by rotation of the second elongated tube (162) at the proximal end (20). The rotation may be motorised, for example, by attachment of the drive shaft of an electric motor to the proximal end (20) of the longitudinal member (144). Preferably, slidable cutting element (160) configured for rotation relative to the first (120) and second (140) clamping element i.e. first (120) and second (140) clamping element remain rotationally static. The rotation may be clockwise, counter-clockwise, or may oscillate between the clockwise and counter-clockwise directions.

The second elongated tube (162) may be disposed within a lumen of the first elongated tube (122). The longitudinal member (142) may be disposed within the lumen of the second elongated tube (162). The longitudinal member (142), second elongated tube (162) and first elongated tube (122) may be disposed in co-axial alignment.

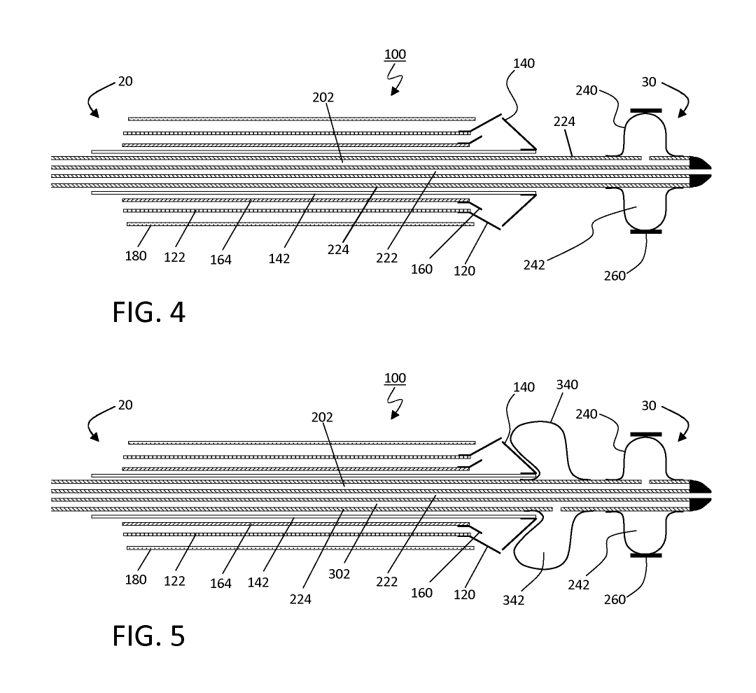

The device (100) may further comprise a heart valve balloon catheter (200) for deployment of an expandable heart valve (260) as shown, for instance, in FIG. 4. The heart valve balloon catheter (200) may be substantially disposed within a lumen of the longitudinal member (144). The heart valve balloon catheter (200) may be slidable within the lumen of the longitudinal member (144). The heart valve balloon catheter (200) typically comprises an inflation lumen (202) extending towards a proximal end of the catheter (200). The inflation lumen is bound by a wall of an inflation tubing (224). The inflation lumen (202) is in fluid communication with a lumen (242) of an expandable balloon (240). The expandable balloon (240) expands or contracts responsive to fluid pressure in the inflation lumen (202). The heart valve balloon catheter (200) further comprises a guidewire lumen (222).

The device (100) may further comprise a centering balloon (340) as shown, for instance, in FIG. 5. This centering balloon (340) is useful for the percutaneous route. The centering balloon (340) assists with correct positioning of the device in the aorta, ensures the device is correctly aligned with the aortic valve, and firmly locks the position of the device during the resection. The centering balloon (340) may further be shaped to limit expansion of the second clamping element (140). The centering balloon (340) may be disposed on a catheter, for instance, on the heart valve balloon catheter (200). The heart valve balloon catheter (200) as mentioned above typically comprises a heart valve balloon inflation lumen (222) extending towards a proximal end of the catheter (200) for inflation of the heart valve balloon (240); it may further comprise the expandable centering balloon (340) and a separate centering balloon inflation lumen (302) extending towards a proximal end of the catheter (200) for inflation of the centering balloon (340). The centering balloon inflation lumen (302) is bound by a wall of an inflation tubing (224). The centering balloon inflation lumen (302) is in fluid communication with a lumen (342) of an expandable centering balloon (340). The expandable centering balloon (340) expands or contracts responsive to fluid pressure in the centering balloon inflation lumen (302).

The device may be provided at the proximal end with a handle for gripping by the user (e.g. surgeon). The first elongated tube (124) may be disposed in fixed relation to the handle. The device (100) is preferably configured for excision of a human heart valve. The device (100) is a surgical device.

The device (100) may be configured for access to the heart valve via open heart surgery. The first (120) and second (140) clamping elements and the slidable cutting element (160) may each be radially non-expandable, as shown, for instance, in FIGS. 1 and 2.

The first (120) and second (140) clamping elements and the slidable cutting element (160) may each be controllably radially expandable. This allows the same device (100) to be used for a variety of different heart valve sizes. The respective radial dimensions may be lockable.

Controllable radial expansion of the first (120) and second (140) clamping elements and the slidable cutting element (160) may be achieved using, for instance, using an inflatable balloon to control a radial dimension.

According to one aspect, the slidable cutting element (160) is provided on an inflatable balloon; the radial dimension of the balloon determines the radial dimension of the cutting edge (162). According to another aspect, the slidable cutting element (160) is conical or frustoconical; the maximum radial dimension is determined by an extent of protrusion from the first elongated tube (124) while force is applied in an outward radial direction inside the conical slidable cutting element (160), for instance by an inflatable balloon, to fix the minimum radial dimension of the conical slidable cutting element (160) i.e. to resist a reduction in the radial dimension by an exterior application of radial force in an inward direction.

The device (100) may be configured for access to the heart valve via a percutaneous route i.e. via the vasculature. The first (120) and second (140) clamping elements and the slidable cutting element (160) may each be radially expandable to reduce their radial dimension during passage through the vasculature, as shown, for instance, in FIGS. 3A and 3B. The first (120) and second (140) clamping elements and the slidable cutting element (160) may each have an open (FIG. 3A) and closed (FIG. 3B) configuration.

In the closed configuration, each of the first (120) and second (140) clamping elements and the slidable cutting element (160) has a narrower profile compared with that in the respective open configurations. In the closed configuration, each of the first (120) and second (140) clamping elements and the slidable cutting element (160) is able to pass substantially unhindered through the lumen of a delivery catheter (180). Each of the first (120) and second (140) clamping elements and the slidable cutting element (160) is capable of expanding from a closed (e.g. FIG. 3B) configuration to an open (e.g. FIG. 3A) configuration; this is typical for deployment through a delivery catheter where each of the first (120) and second (140) clamping elements and the slidable cutting element (160) remains closed while the delivery catheter is advanced, and expands during deployment. Each of the first (120) and second (140) clamping elements and the slidable cutting element (160) is also capable of contracting from an open configuration to a closed configuration for when they are withdrawn back into the delivery catheter.

The slidable cutting element (160) may be configured to compress or compact or fold the excised heart valve by contraction of the cup-shaped body from the open to the closed state. The contraction is typically radial. Compression or compaction or folding forces may be transmitted to the slidable cutting element (160) by its withdrawal into the lumen of the first elongated tube (124). It will be appreciated that other mechanisms for compression or compaction are envisaged by the present invention.

Preferably in the closed configuration the first clamping element (120) has a maximum outer transverse-cross-sectional diameter of 0.5 cm, 0.6 cm, 0.7 cm, 0.8 cm, 0.9 cm, 1 cm, 1.1 cm, 1.2 cm, 1.3 cm, 1.4 cm, 1.5 cm or a value in the range between any two of the aforementioned values, preferably between 0.8 cm to 1.1 cm, most preferably about 0.9 cm. The maximum outer transverse-cross-sectional diameter of the second clamping element (140) is preferably less than that of the first clamping element (120) in the closed configuration. The maximum outer transverse-cross-sectional diameter of the slidable cutting element (160) is preferably less than that of the second clamping element (140) in the closed configuration.

Preferably in the open configuration each of the first clamping element (120) has a maximum outer transverse-cross-sectional diameter of 1.5 cm, 1.6 cm, 1.7 cm, 1.8 cm, 1.9 cm, 2 cm, 2.1 cm, 2.2 cm, 2.3 cm, 2.4 cm, 2.5 cm, 2.6 cm, 2.7 cm, 2.8 cm or a value in the range between any two of the aforementioned values, preferably between 2 cm to 2.5 cm, most preferably about 2.2 cm. The maximum outer transverse-cross-sectional diameter of the second clamping element (140) is preferably less than that of the first clamping element (120) in the open configuration. The maximum outer transverse-cross-sectional diameter of the slidable cutting element (160) is preferably less than that of the second clamping elements (140) in the open configuration.

Each of the first (120) and second (140) clamping elements and the slidable cutting element (160) may be radially expandable. One or more of the first (120) and second (140) clamping elements and the slidable cutting element (160) may be longitudinally expandable. One or more of the first (120) and second (140) clamping elements and the slidable cutting element (160) may be non-longitudinally expandable. Preferably, each of the first (120) and second (140) clamping elements and the slidable cutting element (160) is radially expandable and non-longitudinally expandable. The size of each of the first (120) and second (140) clamping elements and the slidable cutting element (160) in the open configuration may be adjustable. Each of the first (120) and second (140) clamping elements and the slidable cutting element (160) may be self-expanding from the closed configuration to the open configuration; in other words, when it is sheathed using a constricting over sheath, each of the first (120) and second (140) clamping elements and the slidable cutting element (160) is in a closed configuration. When unsheathed, each of the first (120) and second (140) clamping elements and the slidable cutting element (160) expands to the open configuration. Such a sheath may be, for instance, a delivery catheter (180), a first elongate tube (124), the first clamping element (120) or a lasso.

Expansion and/or contraction of each of the first (120) and second (140) clamping elements and the slidable cutting element (160) may be actuated by an expansion actuation mechanism. Such mechanism may utilize sheathing/unsheathing, or the like. It will be appreciated that the expansion is reversible i.e. each of the first (120) and second (140) clamping elements and the slidable cutting element (160) is capable of expansion and contraction.

One or more, preferably all of the first (120) and second (140) clamping elements and the slidable cutting element (160) may be elongate. The outer shape of one or more, preferably all of the first (120) and second (140) clamping elements and the slidable cutting element (160) in the open configuration preferably may be at least a partly conical, most preferably frustoconical. The wide base of the cone preferably provides the open end, while the tip or truncated tip forms the closed end of the slidable cutting element (160).

Where the slidable cutting element (160) is conical, the wide base of the cone preferably provides the open end of the slidable cutting element (160), while the tip or truncated tip forms the closed end. Where the second clamping element (140) is conical, the wide base of the cone preferably provides the open end of the cap (146) of the second clamping element (140), while the tip or truncated tip forms the closed end of the cap (146). Where the first clamping element (120) is conical, wide base of the cone preferably provides the first clamping element (120). Other outer shapes in the open configuration are envisaged for instance, cylindrical, barrel, bullet, rivet and the like. The outer shape in the closed configuration is preferably cylindrical, but other shapes are envisaged such as barrel, bullet, rivet and the like.

In one embodiment, one or more, preferably all of the first (120) and second (140) clamping elements and the slidable cutting element (160) is formed from a self-expanding cone. It is preferably formed from a shape memory material such a NiTinol. In the open configuration the self-expanding cone forms a conical shape. In the closed configuration, the self-expanding cone forms a cylindrical shape. In the native state, no application of force is required to maintain the open configuration. The self-expanding cone is preferably conical in the native state. When a radial force is applied, the self-expanding cone may be moved radially inwards, thereby reducing the diameter towards the closed configuration.

The self-expanding cone may be made using processes similar to making a self-expanding stents. The self-expanding cone may be made from a flat, perforated structure that is subsequently rolled to form the conical structure that is woven, wrapped, drilled, etched or cut to form passages. The flat structure is typically the arc of an annulus. Self-expanding cone may be braided, from flexible metal, such as special alloys, from NiTinol, or from phynox. Self-expandable cone made from NiTinol may be laser cut.

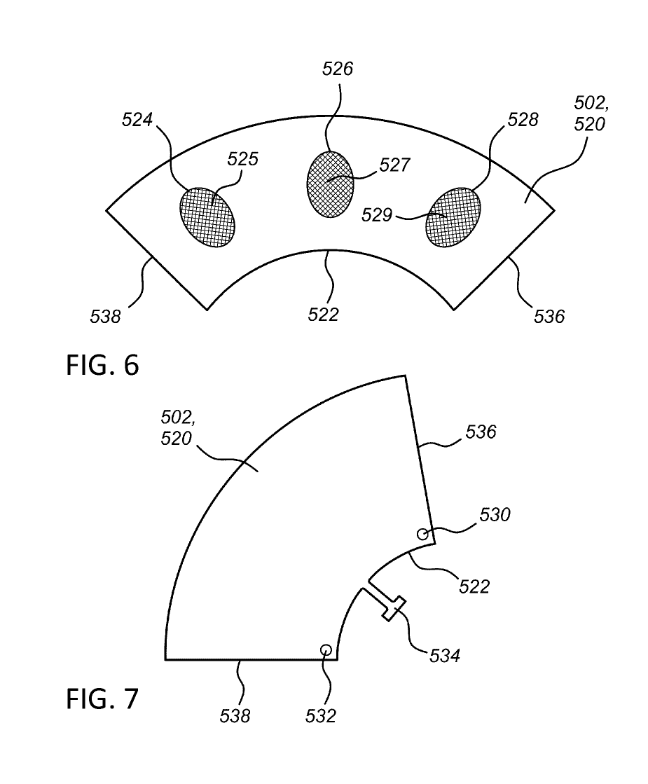

In one embodiment, depicted for instance in FIG. 6, the self-expanding cone (500) has a wall (502) optionally provided with one or more apertures (524, 526, 528). It will be appreciated that self-expanding cone (500) has a proximal (20) and distal (30) end, corresponding to the proximal (20) and distal (30) end of the device (100). The self-expanding cone (500) contains a longitudinal slit that cuts across the cone wall (502). The longitudinal slit is preferably in the direction of the central axis (508) of the expandable cone. The longitudinal slit preferably extends from the proximal (20) edge to the distal (30) edge of the self-expanding cone (500). The longitudinal slit is preferably continuous. The longitudinal slit preferably opens the self-expanding cone (500). Preferably, proximal and distal ends of the self-expanding cone (500) are not continuous as a result of the longitudinal slit. The longitudinal slit provides two outer side edges (536, 538), which overlap in the open and closed configurations. The edges (536, 538), slide or pivot relative to each other as the cone transitions from the open to the closed state, and vice versa. The self-expanding cone (500) contracts into the closed configuration by wrapping the wall (502) of the self-expanding cone (500) into a spiral.

The expandable cone (500) is formed from a material able to transmit the requisite clamping or cutting forces to the tissue, and which is able to contract and expand, such as surgical stainless steel or NiTinol. It is appreciated that the use of a shape memory material such as NiTinol, which, in the native state adopts the shape of the (open) cone, would assist in expansion of the self-expanding cone (500) as it is advanced through the delivery catheter (180).

The wall (502) of the self-expanding cone (500) is preferably formed from a sheet of material (520) comprising a geometric shape that is an annulus segment as depicted in FIGS. 5 and 6. The angle of the segment may be equal to or greater than 60 deg, 65 deg, 70 deg, 75 deg, 80 deg, 85 deg or 90 deg, or a value in a range between any two of the aforementioned values, preferably between 70 and 80 deg, more preferably between 75 deg and 80 deg. The inner annular edge (522) of the sheet—that is the smaller curved (arced) edge—is bent into a circle and attached to the first elongate member (52). The outer annular edge of the sheet—that is the larger (arced) curved edge—forms the cutting edge. The outer side (flanking) edges (536, 538)—that is the two edges that the limit the angle of the segment—overlap. In other words, each flanking edge lies adjacent to a wall of the annulus segment. The sheet (520) or wall (502) thereof may contain one or more apertures or windows (524, 526, 528), (FIG. 6) thereby giving the wall (502) of the expandable cone (500). They allow fluids to escape during compression or compaction. The apertures or windows, or expandable cone (500) may be disposed with a lining material (525, 527, 529) (e.g. a sheet with a fine mesh). The lining material reduces or prevents the leakage of debris or particulate matter from receptacle void. The lining material is preferably a polymeric fine mesh. The wall (502) of the expandable cone may comprise two holes (530, 532) located adjacent to the outer side edges (536, 538) of the annulus segment and to the inner annular edge (522) (FIG. 7). When the annulus segment is bent into a cone, the two holes (530, 532) align and act as a pivot point for the expansion (fanning-out) and contraction of the expandable cone (500). The aligned holes (530, 532) may be secured using a rivet or other means. The sheet of material (520) may further be provided with a tab (534) that extends from the inner annular edge (522); such tab may be aligned with a reciprocating groove in the first elongate member (52) to anchor or secure the expandable cone 500 in relation to the first elongate member (52). In addition, or alternatively, the tab may transmit torque. In a preferred embodiment, the tab has a T-shape, the base of the T extending from the inner annular edge (522).

In one embodiment, one or more of the first (120) and second (140) clamping elements and the slidable cutting element (160) is each formed from a plurality of elongate strips arranged around a ring, each elongate strips pivoted at one and the same end and the other end providing respectively the first (120) and second (140) clamping elements and the slidable cutting element (160).

In the open state the pivoted elongate strips form a conical shape. A pivoted elongate strip may take the form of a compliant member fixed at one end in relation to the ring, the other end forming an open conical shape in the native state. A pivoted elongate strip may alternatively take the form of a rigid member fixed at one end in relation to the ring using a hinge joint. In the native state, the hinged strip may adopt a position contributing to the open conical shape using a spring. In the native state, no application of force may be required to maintain the open configuration. When a radial force is applied, the pivoted strip may be moved radially inwards, thereby reducing the diameter towards the closed configuration.

A movement limiter (a stop) may be provided, which restricts the opening of the pivoted elongate strips to a certain size. The limiter may comprise interconnections between adjacent elongate strips. Alternatively, the limiter may comprise loop of variable diameter that passes around the outside of the cone thereby stopping the cone from opening past a certain diameter.

The diameter may be controlled by the operator from the proximal (20) end, for instance, by feeding a length of wire to the loop “lasso” from the proximal end. Alternatively, the limiter may comprise loop of fixed diameter that passes around the outside of the cone thereby stopping the cone from opening beyond a certain diameter. By displacing the loop in a longitudinal manner, the size of the opening can be controlled by the operator from the proximal (20) end. Alternatively or additionally, the size of the cone in the open configuration may be set, for instance, by the extent the cone is advanced forward from a sheath (e.g. delivery catheter (180), first elongated tube (124)). An elongate strip may be made from any biocompatible material, for instance, stainless steel, titanium, NiTinol, or from a polymeric substance such as polycarbonate.

The present invention also relates to a method for excision of a heart valve using a device (100) as described herein comprising the steps:
positioning the first (120) and second (140) clamping elements either side of the heart valve,
closing the first (120) and second (140) clamping elements together thereby clamping the heart valve in an annular clamping region (166),
actuating the slidable cutting element (160), thereby excising the clamped heart valve

Closing the first (120) and second (140) clamping elements together thereby clamping the heart valve in an annular clamping region (166) may form a first closed container. After excision of the clamped heart valve, the device (100) may be withdrawn while the first closed container is closed; thus additional debris is removed from the subject

1. A device (100) for excision of a heart valve comprising:
a first (120) and second (140) clamping element in mutual sliding relation, each having an annular clamping surface (122, 142) which annular clamping surfaces (122, 142) mutually co-operate to form an annular clamping region (166) configured for clamping a heart valve annularly, and
a slidable cutting element (160) slidable and rotatable with respect to the annular clamping region (166) configured to circularly excise the heart valve,
wherein the slidable cutting element (160) is displaceable within an annulus of the annular clamping zone region (166).

2. The device (100) according to claim 1, wherein the second clamping element (140) comprises a cap (146), which cap (146) comprises a void space configured for retention of tissue debris.

3. The device (100) according to claim 1, wherein the first clamping element (120) comprises a hollow tubular member (121).

4. The device (100) according to claim 1, wherein the slidable cutting element (160) and second clamping element (140) mutually co-operate to form a first closed container for retention of tissue debris.

5. The device (100) according to claim 4, wherein the first (120) and second (140) clamping elements mutually co-operate to form a second closed container for retention of tissue debris, wherein the first container is disposed within the second container.

6. The device (100) according to claim 1, wherein one of the first (120) or second (140) clamping elements is configured to fittingly receive at least part of the other of the first (120) or second (140) clamping elements.

7. The device (100) according to claim 1, wherein the slidable cutting element (160) is disposed on a circular edge of a cup-shaped body configured for retention of tissue debris.

8. The device (100) according to claim 1, wherein the first clamping element (120) is attached to a first elongated tube (124), the slidable cutting element (160) is attached to a second elongated tube (164) arranged within a lumen of the first elongated tube (124), and the second clamping element (140) is attached to a longitudinal member (144) arranged within a lumen of the second elongated tube (164).

9. The device (100) according to claim 1, further comprising a heart valve balloon catheter (240) for deployment of an expandable heart valve (260).

10. The device (100) according to claim 9, wherein the first clamping element (120) is attached to a first elongated tube (124), the slidable cutting element (160) is attached to a second elongated tube (164) arranged within a lumen of the first elongated tube (124), and the second clamping element (140) is attached to a longitudinal member (144) arranged within a lumen of the second elongated tube (164) and wherein the heart valve balloon catheter (240) is substantially disposed within a lumen of the longitudinal member (144).

11. The device (100) according to claim 1, wherein the first (120) and second (140) clamping elements and the slidable cutting element (160) are each expandable in a radial dimension.

12. The device (100) according to claim 11, wherein the first (120) and second (140) clamping elements and the slidable cutting element (160) are each radially expandable compliant members biased in an open configuration.

13. The device (100) according to claim 11, further comprising a deployment catheter (180), wherein the first (120) and second (140) clamping elements and the slidable cutting element (160) are retractable into a lumen of the deployment catheter (180).

14. The device (100) according to claim 1, wherein the first (120) and second (140) clamping elements and the slidable cutting element (160) are each radially non-expandable.

15. The device (100) according to claim 1, wherein the first (120) and second (140) clamping elements and the slidable cutting element (160) are controllably radially expandable, optionally the respective radii being lockable.x
